(19) United States
(12) Patent Application Publication
(10) Pub. No.: US 2009/0170945 A1
(43) Pub. Date: Jul. 2, 2009

(54) PREGABALIN SALTS

(76) Inventor: Raymond J. H. Westheim, Oss (NL)

Correspondence Address: SYNTHON IP INC, 7130 HERITAGE VILLAGE PLAZA, STE 202, GAINESVILLE, VA, 20155, US

(21) Appl. No.: 12/339,922

(22) Filed: Dec. 19, 2008

Related U.S. Application Data
(60) Provisional application No. 61/009,017, filed on Dec. 21, 2007.

(51) Int. Cl. A61K 31/197; C07C 229/08

(52) U.S. Cl. 514/561; 562/553

(57) ABSTRACT

Pregabalin besylate and pregabalin tosylate are pharmaceutically acceptable acid addition salts of pregabalin that are isolatable in solid state, particularly crystalline state, sufficiently soluble in water, and stable both in the solid state and in the solution.

This application claims the benefit of priority under 35 U.S.C. § 119(e) from prior U.S. Provisional Application Ser. No. 61/009,017, filed Dec. 21, 2007; the entire contents of which are incorporated herein by reference.

### BACKGROUND OF THE INVENTION

The present invention relates to pharmaceutically acceptable salts of pregabalin and pharmaceutical compositions containing the same.

Pregabalin, S-(+)-4-amino-3-(2-methylpropyl)butanoic acid of the formula (1)

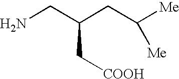

is a structural analogue of GABA. The molecule has one centre of optical activity. Pregabalin acts as a modulator of voltage-gated calcium channels in the CNS, having the potential to treat neuropsychiatric disorders and pain. It is currently marketed as a capsule for oral administration under the brand name LYRICA®, by Pfizer Inc.

Pregabalin is an amino acid, i.e., it contains both a basic amino group and an acidic carboxy group, and thus can exist as a zwitterion (i.e., in a state where the carboxyl group is deprotonated and the primary amine moiety is protonated). Pregabalin may therefore form salts with both acids and bases. The pKa values are 4.2 and 10.6, respectively.

Pharmaceutically acceptable salts of pregabalin formed with organic or inorganic acids or bases were suggested in a patent family arising from WO 93/23383 (e.g., EP 641 330). While no salts were actually made, it was disclosed that “the acid addition salts [ ]are prepared either by dissolving the free base in aqueous or aqueous alcohol solution or other suitable solvents containing the appropriate acid and isolating the salt by evaporating the solution. Examples of pharmaceutically acceptable salts are hydrochlorides, hydrobromide, hydrosulfates, etc., as well as sodium, potassium, and magnesium, etc., salts.” (See WO 93/23383 at pages 6-7.)

It appears that only two pregabalin acid addition salts have been made: pregabalin hydrochloride (WO 2005/041927) and pregabalin (S)-mandelate (WO 96/40617, which also published as U.S. Pat. No. 5,637,767).

Pregabalin hydrochloride was prepared only as an aqueous solution in situ, not as an isolated solid form (see WO 2005/041927 at, e.g., paragraph [0074] et seq. and paragraph [00128]). Pregabalin (S)-mandelate has been described in WO 96/40617 as being isolated in a solid form by a process of resolving a mixture of pregabalin enantiomers (see WO 96/40617 at pages 12-13 and 22). As apparent from the disclosure in WO 96/40617, the compound easily hydrolyzes in water to liberate the free pregabalin: this is advantageous in the resolution process, but not in pharmaceutical applications.

Although pregabalin is used as the free form in the marketed pharmaceutical, it is desirable to provide an alternative pharmaceutically acceptable acid addition salt thereof. In particular, the free form of pregabalin has somewhat limited solubility in water (less than 36 mg/ml). A water soluble salt could be more convenient for use in aqueous pharmaceutical formulations for parenteral or oral applications than the free form. For such purpose, the salt should be sufficiently soluble in water (greater than 100 mg/ml). Furthermore, the salt should be sufficiently stable, both in the solid state and in the solution, and it should be obtainable in solid, preferably crystalline, form.

Thus, it would be desirable to find a solid, particularly crystalline, acid addition salt of pregabalin that would be sufficiently soluble in water and sufficiently stable against formation of impurities.

### SUMMARY OF THE INVENTION

It has been found that the benzene sulfonate salt (hereinafter referred to as the besylate salt) and p-toluene sulfonate salt (hereinafter referred to as the tosylate salt) of pregabalin are compounds with high aqueous solubility that have a number of advantages over the other acid addition salts of pregabalin, and additionally, it has been found that they are suitable for the preparation of pharmaceutical formulations of pregabalin, particularly liquid formulations for oral and parenteral applications.

Accordingly, a first aspect of the present invention relates to a pregabalin sulfonic acid salt selected from pregabalin besylate and pregabalin tosylate. The salt can be in a solid state, preferably a crystalline state, and is preferably an anhydrate free from solvent.

In an embodiment, the crystalline pregabalin besylate exhibits an XRPD pattern substantially corresponding to FIG. 1, a melting point (capillary) in the range of 135.6-139.8° C., and/or a DSC melting endotherm in the range of 136-138° C. In another embodiment, the crystalline pregabalin tosylate typically exhibits an XRPD pattern substantially corresponding to FIG. 2, a melting point (capillary) in the range of 132.4-134.0° C., and/or a DSC melting endotherm in the range of 132-134° C.

Another aspect of the present invention relates to a pharmaceutical composition comprising the salt of pregabalin and a pharmaceutically acceptable diluent or carrier. The pharmaceutical composition can be in a solid form, but is preferably in a liquid form.

A further aspect of the present invention relates to a process for making the salt of pregabalin, comprising precipitating the salt from an inert solvent containing pregabalin and the corresponding sulfonic acid dissolved therein.

A still further aspect of the present invention relates to a process, which comprises reacting pregabalin and a pharmaceutically acceptable sulfonic acid selected from benzene sulfonic acid and p-toluene sulfonic acid in a solvent to form a sulfonic acid salt of pregabalin. Typically, the process further comprises precipitating and isolating the salt from the solvent. The process may also include purifying the isolated salt and/or converting the salt to form pregabalin free base.

Finally, the present invention relates to a method of treating a pregabalin-treatable disease or condition, which comprises administering an effective amount of the pregabalin sulfonic acid salt selected from pregabalin besylate and pregabalin tosylate to a patient in need thereof. The pregabalin-treatable diseases and conditions include treating schizophrenia, epilepsy, pain, etc.

### BRIEF DESCRIPTION OF THE DRAWINGS

FIG. 1 shows an XRPD spectrum of pregabalin besylate.

FIG. 2 shows an XRPD spectrum of pregabalin tosylate.

FIG. 3 shows a DSC scan of pregabalin besylate.

FIG. 4 shows a DSC scan of pregabalin tosylate.

The measurement conditions for the above XRPD spectrums are as follows:

Apparatus: Bruker-AXS D8 vario, Θ/2Θ geometry, reflection mode, Vantec PSD detector
Radiation type: Cu
Radiation wavelengths: 1.54060 Å, primary monochromator used.

The measurement conditions for the above DSC scans are as follows:

Apparatus DSC: Mettler Toledo DSC821e/400, differential scanning calorimeter with a ceramic heat flux sensor.
Atmosphere DSC: Nitrogen, 50 ml/min.
Crucible DSC: Aluminium standard 40 μl, lid pierced.
Heating rate: 10° C./min

### DETAILED DESCRIPTION OF THE INVENTION

The present invention relates to two sulfonic acid salts of pregabalin, namely pregabalin besylate and pregabalin tosylate. These salts can be isolated in solid state, particularly crystalline state, have good water solubility, and are stable in both the solid state and in solution.

A “pharmaceutically acceptable” salt is a salt in which the acid anion is not toxic to the human body to such an extent that it would affect the safety of the administration of pregabalin. Accordingly, salts such as cyanides are excluded from the present invention as not being pharmaceutically acceptable.

“Pregabalin” as used herein refers to S-(+)-4-amino-3-(2-methylpropyl) butanoic acid; in particular, the pregabalin useful in making the salts of the present invention contains less than 0.5% of the corresponding (R) enantiomer and less than 0.5% of structurally related impurities.

Pregabalin is obtainable by known methods, such as those described in EPB 641 330. In free form, pregabalin is white or almost white crystalline compound, soluble in water at a concentration of less than 36 mg/ml, and poorly soluble in ethanol.

The formation of pregabalin acid addition salts was studied with the following acids:

hydrochloric, sulfuric, and phosphoric acid.

methane sulfonic, ethane sulfonic, ethane 1,2-disulfonic, benzene sulfonic, p-toluene sulfonic, D(+)-10-camphorsulfonic, naphthalene 1-sulfonic, and naphthalene 1,5-disulfonic acid.

maleic, fumaric, malonic, glycolic, 1-hydroxy-2-naphthoic, pamoic, (S)-mandelic, L-tartaric, and L-malic acid.

natural L-amino acids, taurine, and saccharine.

The selection of advantageous acid addition salts of pregabalin that would be suitable for pharmaceutical applications, particularly in aqueous solutions, was based on the following criteria.

One criteria is that the salt should be isolatable as a well defined solid, particularly a crystalline material. This is advantageous for handling, charging, and storing the material. Because pregabalin is a zwitterion, the acid for making the salt should be a sufficiently strong acid (in terms of pKa), as weaker acids can hardly form defined salts due to the fact that the acidic carboxyl group of the pregabalin competes with the salt formation. Preferably, the pKa value of the acid is more than 2 values below that of the acidic group of pregabalin.

Another criteria is that the salt is preferably non-hygroscopic. Absorbed films of moisture can act as a vector for hydrolysis and chemical breakdown. The hygroscopic nature of a drug or its salt causes the presence the free moisture, which is often responsible for instability.

A further criteria is that the formation and storage of pregabalin salts should not be accompanied by the formation of impurities. Some acids may react with the amino-group of the pregabalin to yield side products. Additionally, it is known that the molecule of pregabalin is susceptible to the formation of an impurity due to the intermolecular condensation of both reactive groups, particularly in solutions. Such an impurity, a lactam (4-isobutylpyrrolidin-2-one) of the formula (2)

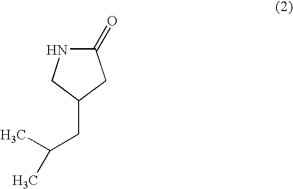

is much more toxic then the “open-ring” form. Acid addition salts of pregabalin, in which the amino group is protonized, may generally be more susceptible to forming this lactam impurity, as the lactam is preferentially formed under acidic pH. An acid forming a salt with low tendency to impurities, and particularly the lactam impurity, is desirable. The salts should be also stable towards oxygen and water to minimize the formation of hydrolytic and oxidative impurities.

A still further criteria is that the salt should be sufficiently soluble in water to allow the preparation of sufficiently concentrated solutions containing a therapeutically effective amount of the pregabalin drug. The conventional dose used in the administration of pregabalin capsules is from 25 to 300 mg per unit. Accordingly, the aqueous solubility of the salt should be greater than 0.1 g/ml, and preferably greater than 0.5 g/ml, calculated as pregabalin free form. The salt should be freely soluble under ambient conditions, i.e., not due to exposing the solution to enhanced temperature.

Consequently, several pharmaceutically acceptable salts of pregabalin were made and evaluated using these criteria. It has been found that while many of the salts of acids outlined above satisfy some of these criteria, none satisfy them all except the besylate and tosylate salts of the present invention.

The ability to form a defined solid, particularly crystalline, salt material was investigated using the pregabalin salt formation process disclosed in the EP 641 330.

Specifically, pregabalin and an equivalent of an acid were dissolved in an aqueous solvent to provide a solution, and the solvent was removed, e.g., by freeze drying. If such process failed, alternate solvents were tested. Alcohol as a solvent has been found to be an unsuitable solvent (generally, strong acids, e.g. sulfonic acids, may catalyze the reaction of pregabalin with the alcohol to form an ester). From the acids studied, many were not able to form solid salts and as a result were excluded from further studies.

For example, from inorganic acids, pregabalin hydrogen sulfate and pregabalin phosphate could not be obtained as a suitable solid. Pregabalin hydrogen sulfate already melted above 40° C., whereas pregabalin phosphate remained a resin. In combining pregabalin with ethane sulfonic acid and with D(+)-10-camphorsulfonic acid, oils were observed which failed to crystallize. No crystalline salt form could be obtained with malonic acid, glycolic acid, taurine, saccharine, and pamoic acid. As discussed above, weak acids are not preferred. Some of them may form a solid product with pregabalin (e.g., (S)-mandelic acid, L-tartaric acid and L-malic acid), but the product is rather a solid co-crystal of pregabalin and the acid having hydrogen bonds instead of ionogenic bonds. Attempts to prepare salts of pregabalin with natural amino acids also failed. The solid product, if obtained, was only a physical mixture or a co-crystal of both components. In case of 1-hydroxy-2-naphthoic acid, only a free acid precipitated with no salt formation.

Pregabalin salts that were preparable in solid state were further tested according to the remaining three criteria.

Hygroscopicity of solid state pregabalin salts that passed the salt formation criterion were tested under storage at ambient and humid (40 C, 75% RH) conditions. Specifically, pregabalin hydrochloride was prepared as a solid crystalline material, which is well soluble in water, but was found to have a hygroscopic tendencies, particularly at humid conditions, to such an extent that it liquefied (dissolved in the absorbed water). The same properties were exhibited by the hydrobromide salt. The naphthalene 1-sulfonate salt was very hygroscopic as it took water up even during weighing, and this salt was formed in a non-stoichiometric amount, i.e., as a badly defined product. The ethane-1,2-disulfonic acid salt proved to be very hygroscopic with fast water uptake during exposure to air, i.e., storage in air (R.T.) for 36 days resulted in the formation of a dihydrate. Also, pregabalin mesylate was very hygroscopic under humid conditions and prolonged storage at 40 C.

Formation of impurities during the salt forming process was tested by analyzing the samples by HPLC after the salt formation. Salts were also subjected to stability studies in the solid state; particularly, they were subjected to storage at 40 C and 75% RH in the open dish.

Some salts made with acid anions are formed with by-products, for instance maleate and fumarate salts. The inherent instability of these salts may be explained by an easy reaction of the primary amino group with the double bond in the acid. The crystalline hydrogenmaleate salt could only be obtained reasonably pure by preparation in methanol, evaporation to dryness, and recrystallization from di-isopropyl ether (IPE) with a small amount of methanol. Pregabalin mandelate was unstable in aqueous solutions, and resulted in the formation of the lactam impurity. Pregabalin besylate, pregabalin 1,5-naphthalene disulfonate (napadisylate), pregabalin tosylate, and pregabalin mesylate were stable during salt formation. However, the napadisylate salt had a tendency to form hydrates and/or solvates and was unstable during prolonged storage on an open dish. The mesylate salt was extremely hygroscopic during prolonged storage.

Solubility of the formed salts was also tested. All isolated solid state pregabalin salts were soluble in water in the desired concentration range. The solubility of most of the pregabalin salts was higher than that of the free base. The least soluble salt was pregabalin mandelate (having a solubility of about 100 mg/ml), followed by pregabalin L-tartrate (having a solubility of about 200 mg/ml) and napadisylate (having a solubility of about 300 mg/ml). All remaining studied salts (hydrochloride, hydrobromide, besylate, tosylate, L-malate) had solubility greater than 500 mg/ml

In conclusion, the only salts among those tested that satisfy all four criteria studied (i.e., isolatable as a well defined salt, non-hygroscopic, no formation of impurities, and sufficient water solubility) are pregabalin besylate and pregabalin tosylate.

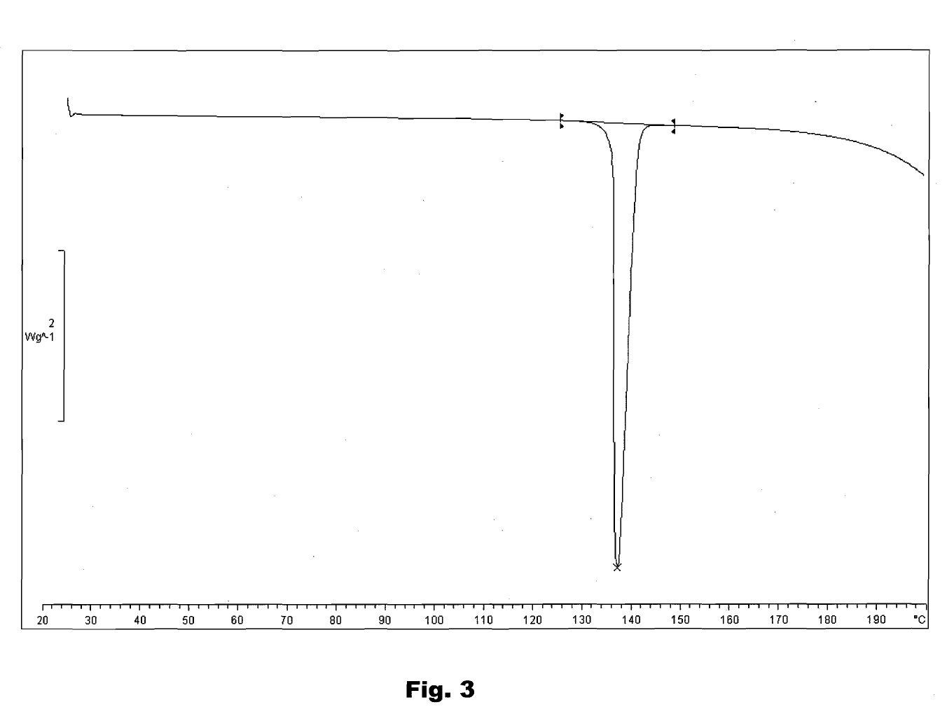

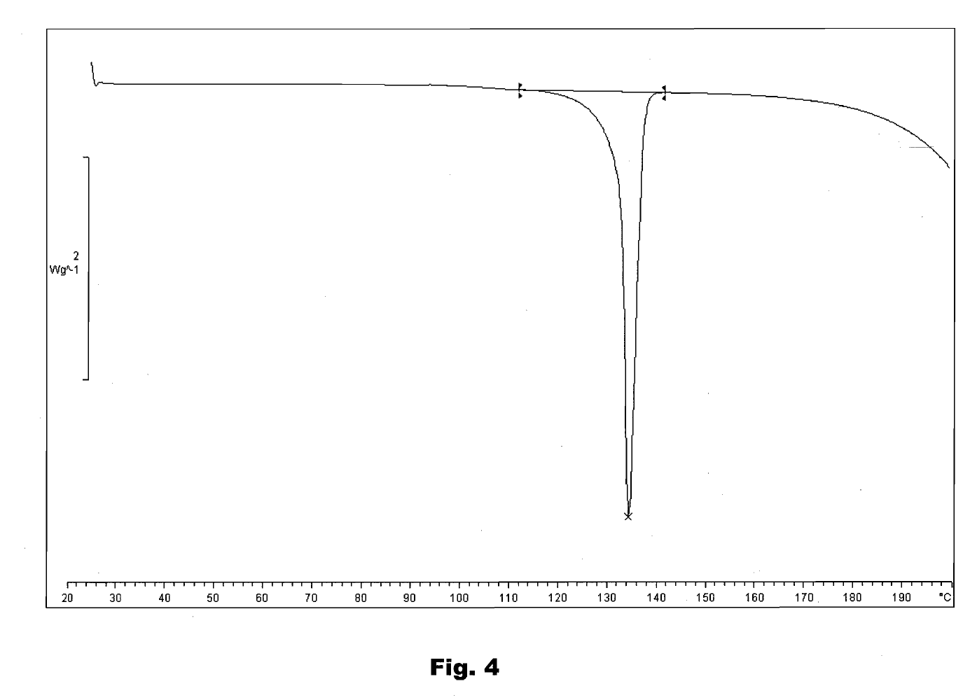

In solid form, pregabalin besylate is a white crystalline powder, with a melting point (capillary) typically in the range 135.6-139.8° C. Under DSC (see FIG. 3) the melting endotherm is typically around 136-138° C. Likewise, pregabalin tosylate is a white crystalline powder with a melting point (capillary) typically in the range 132.4-134.0° C. Under DSC (see FIG. 4) the melting endotherm is typically around 132-134° C. For clarity, the pregabalin besylate and pregabalin tosylate include not only solid forms but also other states especially dissolved forms. This is true even if the dissolved form results in the ions being completely dissociated and hence in a strict technical sense not a salt. For simplicity, however, the term “salt” as used herein carries its liberal or casual meaning and embraces the solid, liquid, dissolved forms, etc. of the salt compound.

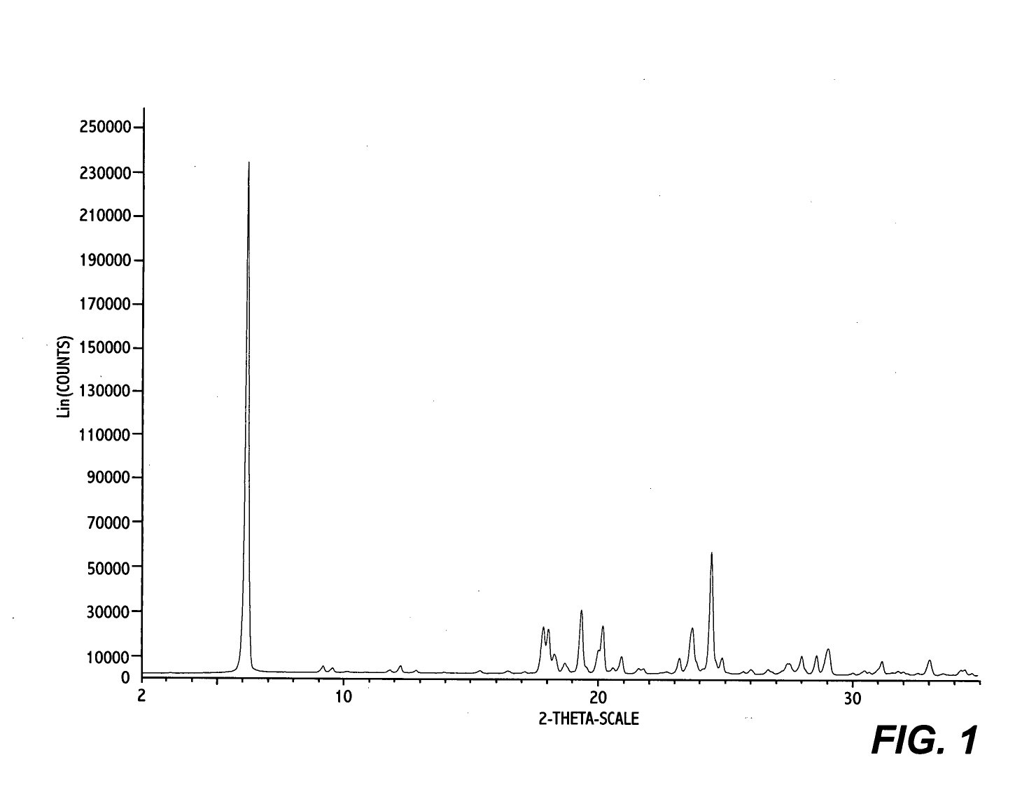

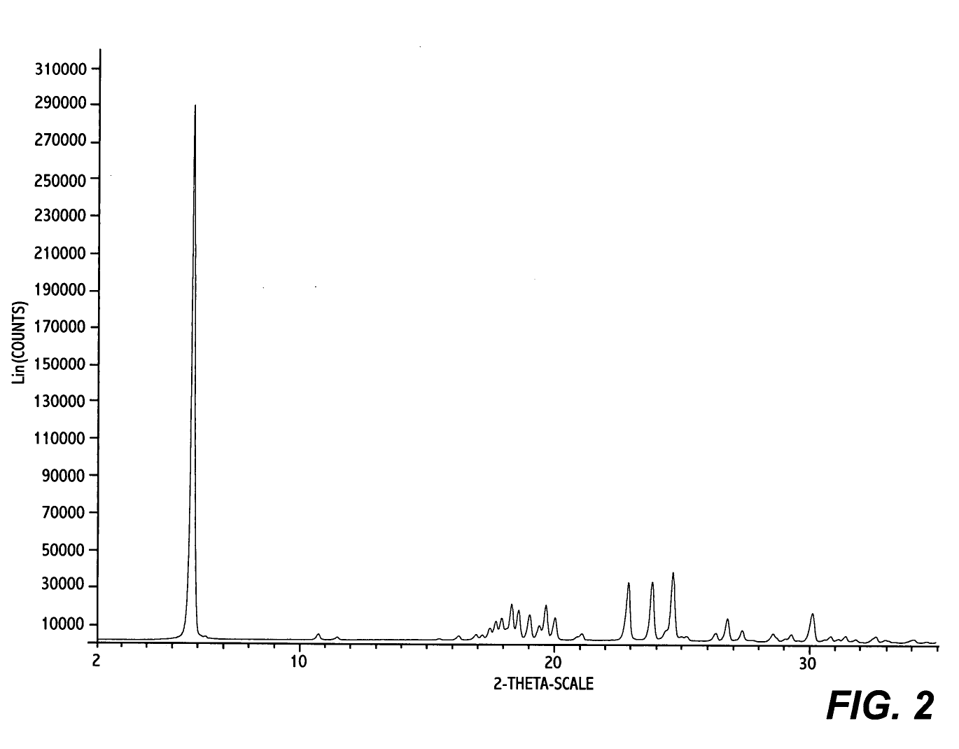

The XRPD spectra of pregabalin besylate and pregabalin tosylate exhibit well defined diffraction peaks confirming the crystalline character of the respective salts, for example as shown in FIG. 1 and FIG. 2, respectively. Characteristic peaks for pregabalin besylate are at angles of about 6.15, 17.86, 18.06, 19.37, 20.96, 23.74, 24.51, 24.93, 28.66, 29.12, +/−0.10° 2θ. Characteristic peaks for pregabalin tosylate are at angles (2θ) of about 5.74, 17.69, 17.92, 18.58, 19.40, 19.66, 20.02, 22.91, 23.85, 24.67, 26.80, 30.15 +/−0.10°2θ. For clarity, these peak angles were determined using the measurement conditions described above for FIGS. 1 and 2. Pregabalin besylate and pregabalin tosylate compounds exhibiting XRPD patterns that substantially correspond with FIGS. 1 and 2, respectively, are specific embodiments of the present invention. The phrases “substantially corresponds” and “substantially as shown in” each encompasses variations caused by different sample preparations, different equipment and/or settings used in measuring, normal experimental error/variation, and small amounts of impurities. Differences in a pattern that are not attributable to these factors indicate that the pattern in question does not “substantially correspond” to the reference pattern.

The pregabalin besylate and pregabalin tosylate each crystallize as anhydrous compounds. NMR demonstrates that the ratio of pregabalin to acid in the crystalline products is essentially 1:1. Solid state forms other than disclosed above, however, are not excluded and fall within the scope of the present invention (such as different polymorphs of the pregabalin besylate and pregabalin tosylate). Thermogravimetric measurements show that both salts are stable up to 160-170° C. Furthermore, the solubility of both salts in water at 25° C. is higher than 500 mg/ml.

In general, the salts of the present invention may be made by dissolving the pregabalin (either in an isolated form or made by a chemical transformation and still a crude product) and the corresponding acid (i.e., benzene sulfonic acid or p-toluene sulfonic acid) in a suitable solvent. The salt forming reaction readily occurs. Typically the salt is then isolated from the solvent, preferably as a solid material.

Suitable solvents include aliphatic cyclic ethers (e.g. dioxane), esters, and ketones (each of the ethers, esters, and ketones having 3 to 12 carbon atoms), and mixtures thereof as well as mixtures with water. The solubility may be enhanced by heating the reaction mixture. The presence of water enhances the solubility of both reagents (i.e., the pregabalin and the corresponding acid) substantially. Due to the high water solubility of the salt, however, the amount of water in the solvent should be minimized prior to the isolation of the salt (e.g. by evaporation of the solvent prior to the isolation), and preferably water is excluded from the solvent entirely. On the other hand, it was shown that the pregabalin salts of the invention are only slightly soluble in aliphatic ethers (such as diisopropyl ether and methyl tert. butyl ether) so that aliphatic ethers may be used as solvents in the salt forming reaction or as antisolvents for the isolation. Furthermore, alcohols as solvents are preferably avoided, as they may be esterified in the presence of the sulfonic acid to yield corresponding esters as byproducts. Thus alcoholic solvents and other non-inert solvents (e.g. solvents that will react under the salt forming conditions) are generally avoided.

The isolation process includes the evaporation of the solvent and/or the precipitation of the salt from the solvent. In this respect, the solvent should also be selected in such a way that the resulting salt is sufficiently insoluble therein and may precipitate therefrom. The insolubility may be enhanced by, for example, cooling the solution, removing a part of the solution, or adding a contrasolvent to the solution. A seeding crystal may be added before or during the precipitation. The salt formation and precipitation phases may follow each other or they may overlap, in whole or in part. Indeed, the precipitation may occur immediately upon formation of the salt in solution thereby providing simultaneous salt forming and precipitation. Such is included within the present invention so along as the formed salt is at least momentarily dissolved in the solvent before it is precipitated. If the salt formation and precipitation phases are divided (e.g., if they occur one after the other in sequence and do not overlap), it may be useful to treat the solution containing the formed salt before precipitation with a suitable adsorption material (such as activated charcoal, etc.) to remove some impurities that may be present therein.

Either the pregabalin or the sulfonic acid may be used in a certain excess (i.e., non-equimolar amounts), and to the extent that the unreacted portion of either starting material remains dissolved, the excess may be removed from the formed salt product. Generally, however, the pregabalin and the sulfonic acid are used in essentially equimolar amounts.

After precipitation, the isolated pregabalin sulfonic acid salt may contain residues of the solvent, which can be removed by careful drying of the salt product, preferably at diminished pressure. After that, the salt product is essentially an anhydrate. That is, the dried pregabalin sulfonic acid salt contains less than 1% of water or other solvent and is essentially stable against environmental moisture (i.e., the dried salt product has almost no tendency to absorb water from the environment). Thus, the compound may be safely stored at ambient conditions.

The pregabalin salts of the present invention may be converted into the pregabalin free form by neutralization, e.g., adding a base. Converting from pregabalin to the salt and back to pregabalin can have beneficial purification effects, especially where different solvents are used; e.g., precipitating the salt from an organic solvent and neutralizing in an aqueous solvent, etc. Further, the pregabalin salts are optionally purified before conversion into the free form.

The salts of the present invention may be formulated with a pharmaceutically acceptable excipient or excipients in various pharmaceutical compositions, including liquid and solid formulations. The pharmaceutical compositions of the present invention include the unit dosage form as well as the intermediate bulk formulations such as solutions, pellets, beads, powder blends, etc. Typically the composition is a finished dosage form also referred to as a unit dose.

Preferred pharmaceutical compositions comprise an aqueous solution for oral or parenteral administration, in which pregabalin salt is used together with suitable pharmaceutical excipients (e.g., a salt, buffer, a co-solvent, a preservative etc.), and may be sterilized by suitable means. The solution, comprising a unit dose amount of pregabalin, may be filled into vials or ampoules. Alternatively, the liquid can be administered as a capsule, e.g., as a liquid gel capsule, where the solid capsule serves as an ingestible container holding the effective dose of the liquid composition therein. The concentration of pregabalin in the unit dose solution may be 1% or higher, such as 1%, 5% or 10%.

In other embodiments, the pharmaceutical compositions can be a solid dosage form, such as a tablet, comprising the solid composition of pregabalin salt and a pharmaceutically acceptable excipient. Pharmaceutically acceptable excipients are well known in the art and include diluents, fillers, binders, lubricants, disintegrants, glidants, colorants, pigments, taste masking agents, sweeteners, plasticizers, and any acceptable auxiliary substances, such as absorption enhancers, penetration enhancers, surfactants, co-surfactants, and specialized oils. The proper excipient(s) are selected based in part on the dosage form, the intended mode of administration, the intended release rate, and manufacturing reliability. Examples of common types of excipients include various polymers, waxes, calcium phosphates, and sugars. Polymers include cellulose and cellulose derivatives such as HPMC, hydroxypropyl cellulose, hydroxyethyl cellulose, microcrystalline cellulose, carboxymethylcellulose, sodium carboxymethylcellulose, calcium carboxymethylcellulose, and ethylcellulose; polyvinylpyrrolidones; polyethylenoxides; and polyacrylic acids including their copolymers and crosslinked polymers thereof, e.g., Carbopol® (B.F. Goodrich), Eudragit® (Rohm), polycarbophil, and chitosan polymers. Waxes include white beeswax, microcrystalline wax, carnauba wax, hydrogenated castor oil, glyceryl behenate, glycerylpalmito stearate, and saturated polyglycolyzed glycerate. Calcium phosphates include dibasic calcium phosphate, anhydrous dibasic calcium phosphate, and tribasic calcium phosphate. Sugars include simple sugars such as lactose, maltose, mannitol, fructose, sorbitol, sacarose, xylitol, isomaltose, and glucose, as well as complex sugars (polysaccharides) such as maltodextrin, amylodextrin, starches including maize, and modified starches.

The pharmaceutical compositions containing the pregabalin salts of the present invention may be used in treating various diseases and conditions, towards which the pregabalin is active. Such pregabalin-treatable diseases and conditions include epilepsy; schizophrenia; neuropsychiatric disorders such as generalized anxiety disorder, panic disorder, and social phobia; and pain including neuropathic pain, fibromyalgia, and acute herpetic and post-herpetic pain. In general, the treatment comprises administering an effective amount (e.g. a pain relieving amount) of the pregabalin salt of the present invention to a patient in need thereof. Liquid pharmaceutical compositions can facilitate finer titration of the dose of the medicament with respect to the patient's response.

The invention will be further described with reference to the following non-limiting examples.

### Example 1

### Pregabalin Benzene Sulfonate

About 0.5 grams of pregabalin was suspended in 100 ml of 1,4-dioxane. The suspension was heated to reflux, while stirring. To the hot suspension, about 0.75 grams of benzenesulfonic acid monohydrate dissolved in 5 ml demi-water was added. As a result, the drug substance dissolved. The clear solution was allowed to cool to room temperature and stirred overnight at room temperature. Then, the solution was concentrated under vacuum, until noticeable crystallization could be observed. The suspension was stirred at room temperature for about 15 minutes. The solid was isolated by filtration over a P3-glass filter (reduced pressure), washed with 1,4-dioxane and vacuum dried overnight at 40° C. Off-white to grayish flake like crystals were obtained. The yield was 470 mg. NMR showed pregabalin: benzenesulfonic acid=1:1. DSC showed melting at around 136-137° C., whereas XRPD showed no peaks of pregabalin free form or benzenesulfonic acid.

### Example 2

### Pregabalin Benzene Sulfonate

5.0 grams of pregabalin and about 6 grams of benzenesulfonic acid monohydrate were dissolved in 60 ml demi-water at room temperature, while stirring. After completed dissolution, the solution was filtered over a P3-glass filter (reduced pressure) to remove possible foreign particles. Then, the solution was rapidly frozen below −70° C. and freeze dried. The obtained white and foamy solid was suspended in 100 ml di-isopropyl ether spiked with a few ml of acetone. The suspension was stirred at room temperature for about 1 day. The solid was isolated by filtration over a P3-glass filter (reduced pressure), washed with di-isopropyl ether and vacuum dried overnight at 40° C. A white to off-white powder was obtained. The yield was 9.54 g. NMR showed pregabalin: benzenesulfonic acid=1:1. DSC showed melting at around 136-138° C., whereas XRPD showed no peaks of pregabalin free form or benzenesulfonic acid.

### Example 3

### Pregabalin p-toluene Sulfonate

About 0.5 grams of pregabalin was suspended in 100 ml of 1,4-dioxane. The suspension was heated to reflux, while stirring. To the hot suspension, about 0.64 grams of p-toluenesulfonic acid monohydrate dissolved in 1-2 ml demi-water was added. As a result, the drug substance dissolved. The clear solution was allowed to cool to room temperature and stirred overnight at room temperature. Then, the solution was concentrated under vacuum, until noticeable crystallization could be observed. The suspension was stirred at room temperature for about 1 hour. The solid was isolated by filtration over a P3-glass filter (reduced pressure), washed with 1,4-dioxane and vacuum dried at room temperature for about 3 days. White, shiny crystals were obtained. The yield was 830 mg. NMR showed pregabalin: p-toluenesulfonic acid=1:1. DSC showed melting at around 135-137° C., whereas XRPD showed no peaks of pregabalin free form or p-toluenesulfonic acid.

### Example 4

### Pregabalin p-toluene Sulfonate

5.0 grams of pregabalin and about 6 grams of p-toluenesulfonic acid monohydrate were dissolved in 60 ml demi-water at room temperature, while stirring. After completed dissolution, the solution was filtered over a P3-glass filter (reduced pressure) to remove possible foreign particles. Then, the solution was rapidly frozen below −70° C. and freeze dried. The obtained white and foamy solid was suspended in 100 ml di-isopropyl ether spiked with a few ml of acetone. The suspension was stirred at room temperature for about 1 day. The solid was isolated by filtration over a P3-glass filter (reduced pressure), washed with di-isopropyl ether and vacuum dried overnight at 40° C. A white to off-white powder was obtained. The yield was 9.95 g. NMR showed pregabalin: p-toluenesulfonic acid=1:1. DSC showed melting at around 132-135° C., whereas XRPD showed no peaks of pregabalin free form or p-toluenesulfonic acid.

### Example 5

### Formulation of Sterile Aqueous Solution of Pregabalin Besylate

Sodium chloride was dissolved in water for injection and propylene glycol was mixed with this solution. The besylate salt of pregabalin was added and, when it had dissolved, further water for injection was added to adjust the volume to give the desired concentration of pregabalin (100 mg/ml). The solution was then filtered through a sterilizing filter and filled into suitable sterile containers, e.g. ampoules, for use in parenteral, e.g. intravenous, administration.

Each of the patents, patent applications, and journal articles mentioned above are incorporated herein by reference. The invention having been described it will be obvious that the same may be varied in many ways and all such modifications are contemplated as being within the scope of the invention as defined by the following claims.

We claim:

1. A pregabalin sulfonic acid salt selected from pregabalin besylate and pregabalin tosylate.

2. The salt of claim 1, which is in a solid state

3. The salt of claim 1, which is in a crystalline state.

4. The salt of claim 3, which is an anhydrate.

5. The salt of claim 2, which is free from solvent.

6. The salt of claim 3, which is pregabalin besylate exhibiting an XRPD pattern substantially corresponding to FIG. 1, exhibiting a melting point (capillary) in the range of 135.6-139.8° C., or exhibiting a DSC melting endotherm in the range of 136-138° C.

7. The salt of claim 3, which is pregabalin tosylate exhibiting an XRPD pattern substantially corresponding to FIG. 2, exhibiting a melting point (capillary) in the range of 132.4-134.0° C., or exhibiting a DSC the melting endotherm in the range of 132-134° C.

8. A pharmaceutical composition, comprising the salt of claim 1 and a pharmaceutically acceptable diluent or carrier.

9. The composition of claim 8, which is a liquid form and contains water as a carrier.

10. The composition of claim 8, which is a solid form.

11. A process, which comprises reacting pregabalin and a sulfonic acid selected from benzene sulfonic acid and p-toluene sulfonic acid in a solvent to form a sulfonic acid salt of pregabalin.

12. The process according to claim 11, which further comprises isolating said salt of pregabalin from the solvent as a crystalline pregabalin besylate or a crystalline pregabalin tosylate.

13. The process of claim 12, wherein the solvent is selected from the group consisting of 3 to 12 carbon aliphatic cyclic ethers, 3-12 carbon esters, 3-12 carbon ketones, mixtures thereof, and mixtures of one or more with water.

14. The process according to claim 13, which further comprises converting the salt of pregabalin to form pregabalin free base.

15. The process according to claim 15, which further comprises purifying the salt of pregabalin before the converting to pregabalin free base.

16. A method of treating a pregabalin-treatable disease or condition, which comprises administering an effective amount of the pregabalin sulfonic acid salt selected from pregabalin besylate and pregabalin tosylate to a patient in need thereof.

17. The method of claim 16, wherein said pregabalin-treatable disease or condition is pain.
